(19) United States
(12) Patent Application Publication
(10) Pub. No.: US 2021/0246094 A1
(43) Pub. Date: Aug. 12, 2021

(54) METHOD FOR PRODUCING FAT ALCOHOL ETHOXYLATES

(71) Applicant: L'Air Liquide, Société Anonyme pour I'Etude et I'Exploitation des Procédés Georges Claude, Paris (FR)

(72) Inventors: Ingo BAUER, Nohfelden (DE); Peter POTSCHACHER, Frankfurt am Main (DE); Guiseppe CUSATI, Frankfurt am Main (DE)

(21) Appl. No.: 17/254,927

(22) PCT Filed: Jun. 12, 2019
(86) PCT No.: PCT/EP2019/025179

§ 371 Date: Dec. 22, 2020

(30) Foreign Application Priority Data
Jun. 22, 2018 (EP) 18400018.0

(51) Int. Cl. C07C 41/09; C07C 41/42; B01J 31/02

(52) U.S. Cl. CPC C07C 41/09; C07C 41/42; B01J 2531/002; B01J 2231/4288; B01J 31/0225

(57) ABSTRACT

The invention relates to a process for preparing fatty alcohol ethoxylates. According to the invention, the fatty alcohol ethoxylates are not obtained by means of the reaction of the fatty alcohols with ethylene oxide, as known from the prior art, but rather by etherification with ethylene glycol, an oligo ethylene glycol or a polyethylene glycol in the presence of an acidic catalyst.

### CROSS REFERENCE TO RELATED APPLICATIONS

This application is a 371 of International Application No. PCT/EP2019/025179, filed Jun. 12, 2019, which claims priority to European Patent Application No. 18400018.0, filed Jun. 22, 2018, the entire contents of which are incorporated herein by reference.

### BACKGROUND

### Field of the Invention

The invention relates to a process for preparing fatty alcohol ethoxylates from fatty alcohols (FA) by using reactants with a low hazardous material potential.

### State of the Art

The preparation of ethoxylates, which can be described with the general formula CₙHₘ(OCH₂CH₂)ₓOH and represent a specific class of nonionic surfactants, is known per se to experts. The properties and use of these surfactants are described in detail in the brochure “Die fleißigen Verbindungen” [The industrious compounds] by the German association TEGEWA e. V., 2014, http://www.tegewa.de/uploads/media/Tensid_Broschuere_2014_deutsch.pdf, retrieved on Aug. 5, 2018.

Ethoxylates are generally obtained by adding ethylene oxide to compounds containing dissociating protons. Used as substrates for the ethoxylation are primarily linear and branched, primary and secondary C₁₂ to C₁₈ alcohols, that is to say, for example, natural and synthetic fatty alcohols. The degree of ethoxylation, i.e. the molar ratio of added ethylene oxide per mole of substrate, varies within wide ranges, in general between 3 and 40, and is selected according to the intended use.

The addition of ethylene oxide to a substrate containing acidic hydrogen is catalysed by bases or (Lewis) acids. Amphoteric catalysts, which were prepared in situ and presumably exist as finely dispersed solids with large surface area, and also heterogenous catalysts have also been described.

The reaction mechanisms of the base-catalysed and acid-catalysed ethoxylation differ, which has an effect on the composition of the reaction products. In the base-catalysed ethoxylation, an alkoxide anion, formed initially by reaction with the catalyst (alkali metal; alkali metal oxide, carbonate, hydroxide or alkoxide), nucleophilically attacks ethylene oxide. The resulting anion of the ethylene oxide addition product can enter into an equilibrium reaction with the alcohol starting material or ethoxylate product, or can react further with ethylene oxide.

The situation is different for the ethoxylation of alcohols. The ether oxygen atoms in alkyl (oligo) glycol ethers increase the acidity of the terminal primary hydroxyl group compared to the initial alcohol; glycol ethers formed in this way thus react preferentially with ethylene oxide and lead to the formation of a mixture of homologous oligo glycol ethers, and unreacted starting alcohol remains in the reaction mixture up to high degrees of ethoxylation. This applies especially to the ethoxylation of secondary alcohols.

If Lewis acids such as boron trifluoride, tin tetrachloride or antimony pentachloride are used as catalysts, homologous distributions approximating the Poisson distribution are obtained.

The handling of ethylene oxide proves to be problematic on account of its reactivity and toxicity. Moreover, ethylene oxide forms flammable vapour mixtures with air in infinite ratios, for which reason handling ethylene oxide requires greater organizational outlay in order to inertize vessels, lines and apparatuses.

### SUMMARY

The object of the invention is therefore that of specifying a process which avoids the stated disadvantages of the processes known from the prior art and in which in particular only reactants having a lower hazardous material potential compared to ethylene oxide are used.

This object is achieved essentially by a process having the features of Claim 1. Further especially preferred configurations of the process according to the invention can be found in the dependent claims.

Process for preparing fatty alcohol ethoxylates, characterized in that the fatty alcohol is reacted with ethylene glycol, an oligo ethylene glycol or a polyethylene glycol in the presence of an acidic catalyst.

Since ethylene glycol is less reactive and therefore easier to transport and to handle, what was investigated was the possibility of obtaining ethoxylated fatty alcohols via an etherification reaction between ethylene glycol and fatty alcohols with elimination of water. It can be assumed that the approach is also conductible with other polyols (condensed ethylene glycols).

By way of a homogeneously, acidically catalysed etherification reaction of a mixture of ethylene glycol and a fatty alcohol, an attempt was made to synthesize an unsymmetrical ether (ethoxylated fatty alcohol) which, due to its amphiphilic structure, could serve as a surface-active product.

### DETAILED DESCRIPTION OF PREFERRED EMBODIMENTS

One preferred configuration of the process according to the invention is characterized in that a homogeneous acidic catalyst is used. The use of a homogeneous catalyst makes it possible to establish good mixing and accordingly good contact between the reactants and the catalyst substance. Problems of mass transfer are avoided in this way.

It has been found to be particularly advantageous for methanesulfonic acid to be used as homogeneous acidic catalyst. Methanesulfonic acid is particularly effective as a catalyst and can be obtained commercially.

In one particular aspect of the process according to the invention, the reaction temperature is between 100 and 160° C., preferably between 130 and 150° C., if methanesulfonic acid is being used as the homogeneous acidic catalyst. In this way operation is conducted at a high reaction temperature and consequently high rates of reaction are achieved, but the temperature remains below the decomposition temperature of methanesulfonic acid, which is above 160° C.

In one further particular aspect of the process according to the invention, the molar ratio of ethylene glycol to the fatty alcohol is between 0.1 and 10 mol/mol, preferably between 0.5 and 5 mol/mol, most preferably between 1 and 3 mol/mol. In this way a favourable compromise is achieved between, firstly, a comparatively high selectivity for the fatty alcohol monoethoxylate and, secondly, a high level of conversion of the fatty alcohol.

One further preferred configuration of the process according to the invention is characterized in that the reaction mixture is cooled after conducting the reaction and is neutralized by adding a base, a light, organic phase separating from a heavy, aqueous phase at the same time. After phase separation is complete, the light, organic phase is separated off from the heavy, aqueous phase by means of a phase separation apparatus which preferably operates by the principle of sedimentation, centrifugation or decantation.

In one further particular aspect of the process according to the invention, the light phase obtained after conducting the phase separation is worked up by means of a thermal separation process to obtain fatty alcohol ethoxylates. In this case the workup is preferably performed by means of distillation or rectification.

It is favourable to use as reactant an oligo ethylene glycol having a number of from 2 to 15, preferably from 2 to 8, ethylene oxide units (—CH₂CH₂O—), since industrially and commercially advantageous nonionic surfactants can be produced in this manner.

### EXAMPLES

### Working and Numerical Examples

Further features, advantages and possible applications of the invention are also apparent from the description of working and numerical examples which follows. All the features described, on their own or in any combination, form the subject-matter of the invention here, irrespective of their combination in the claims or the dependency references thereof.

### Working Example 1

### Reaction

A mixture of 124 g of ethylene glycol (2.0 mol), 130 g of n-octanol (1.0 mol, as a model substance for a fatty alcohol) and 25 g of 77% methanesulfonic acid (corresponding to 19.2 g of MSA; 0.2 mol) as catalyst was heated to 90° C. in a 1 litre round-bottom flask with stirring (magnetic stirrer, 1000 rpm). The methanesulfonic acid dissolved in the process and a homogeneous mixture was established. The glass apparatus was equipped with a Liebig condenser, so that water of reaction formed could be condensed and measured. The condensation temperature was approximately 20° C.

At the start of the experiment, the heating power was set to 90 watts and a nitrogen stream of 500 ml/min was introduced into the mixture, which continued to be agitated, through a gas introduction pipe. In order to operate considerably below the decomposition temperature of the methanesulfonic acid (>160° C.), a maximum reaction temperature in the range of 140 to 150° C. was striven for.

Due to the considerable and persistent formation of condensate, the reaction was carried out over 7 h.

The amount of condensate of 26.1 g obtained at the end of the reaction time was biphasic and consisted of 21.5 g of a lower, aqueous phase and 4.6 g of an upper, oily phase which was not identified further. An octanol odour was not pronounced. The condensed amount of water cannot be viewed as quantitative, as it is not possible to assume quantitative condensation by dint of the nitrogen stream and the condensation temperature.

### Workup of the Reaction Product

After cooling the reaction mixture to approximately 50° C., the entire reaction mixture was neutralized against methyl orange with 37 g of 22% sodium hydroxide solution (corresponding to 29 g of water+8.0 g; 0.2 mol of NaOH). After neutralization, the entire reaction mixture of 283 g was transferred into a separatory funnel. Two phases formed within a few seconds, which, after separation at room temperature (30° C.), was divided into a 156 g oily upper phase and a 127 g lower phase.

Mixtures of in each case 1 ml of phase and 50 ml of water were prepared from both phases and were shaken vigorously. Neither of the two phases exhibited any foam formation.

However, the mixture thus prepared containing the oily upper phase exhibited a clear emulsifying capability, for which reason it was possible to assume that surface-active substances had formed in the reaction mixture and had accumulated in the oily upper phase. 1 ml of octanol was additionally added to the mixture containing the glycolic lower phase and water, and vigorous shaking was performed. No emulsifying capability was apparent.

Thereupon, the oily upper phase was further worked up by distilling off the unreacted fraction of octanol at 120° C. and 15 mbar in a rotary evaporator. 47 g (0.36 mol) of octanol and approximately 2 g of water were obtained as condensate. This distillation did not proceed quantitatively, since 17% of octanol was still detected analytically in the bottoms product (see Table 1). This corresponded to an amount of 18 g, or 0.14 mol, of octanol. Therefore, approximately 0.5 mol (0.36 mol+0.14 mol) of the octanol (corresponding to 50% of the starting amount of 1 mol) have been converted.

After concentration in a rotary evaporator, 107 g of bottoms product were obtained, which was analysed. To this end, an attempt was made to identify the individual components by means of GC-MS and then to quantify them by means of GC-FID. This was only partially successful (see Table 1). Since there were likewise no standards of products and by-products, evaluation was done via the area percentages, and so the presented results can be judged as semi-quantitative.

TABLE 1
Analysis of the worked-up upper phase

| | Retention | | | Estimation of |
|---|---|---|---|---|
| | time | | | boiling range |
| Component | (min) | Area | Area % | (° C.) |
| Ethylene glycol | 15.62 | 143 | 0.9 | 197 |
| 1-Octanol | 19.96 | 2799 | 16.9 | 196 |
| C₁₀H₂₂O₂ | 27.36 | 6688 | 40.4 | 230-240 |
| Unknown | 34.40 | 1547 | 9.3 | 270-280 |
| Dioctyl ether | 36.55 | 3817 | 23.1 | 286-287 |
| Unknown | 40.46 | 287 | 1.7 | 310-320 |
| Unknown | 42.25 | 1096 | 6.6 | ~350 |
| Unknown | 47.25 | 181 | 1.1 | ~360 |
| Sum total | | 16 558 | 100 | |

Approximately 40% of the expected target product ethylene glycol monooctyl ether (C₁₀H₂₂O₂) was identified in the worked-up upper phase. With reference to the measured mass of the upper phase of 107 g, it is possible to provide information about octanol-based selectivity:
0.5 mol of octanol was converted (see above).
Of this, 43.2 g (=40.4%*107 g), or 0.25 mol (molar mass=174.3 g/mol), have been converted to the target product ethylene glycol monooctyl ether, corresponding to an octanol-based selectivity of 50%.
The formation of dioctyl ether was 24.7 g (=23.1%*107 g), or 0.14 mol (molar mass=242.5 g/mol), corresponding to an octanol-based selectivity of 28%.
Approximately 0.11 mol remain as unidentifiable octanol-based compounds (balance to 0.5 mol of converted octanol), corresponding to an octanol-based selectivity of 22%.

### Working Example 2

Molar ratio of dodecanol, ethylene glycol, methanesulfonic acid=1+2+0.3; total charge 500 g.

274.1 g (1.47 mol) of dodecanol were weighed together with 43.3 g of methanesulfonic acid (0.44 mol) into a round-bottom flask and this was placed in a heating sleeve in a fume cupboard. Thereafter, this flask was equipped with a Liebig condenser (water temperature 20° C.), temperature sensor and a gas introduction pipe.

After activating a nitrogen stream of 0.5 l/min and heating (target temperature 150° C.), the reaction mixture was heated under vigorous stirring using a magnetic stirrer. The start of the reaction was defined upon reaching 100° C. From this point in time, a total of 182.6 g of ethylene glycol (2.94 mol) were added continuously (30.4 g/h) within the following 6 hours. The target temperature of 150° C. was reached after 15 min. After complete addition of the ethylene glycol (6 h), the reaction was continued further for an additional hour.

An amount of condensate of 95 g was detected gravimetrically over the entire reaction period of 7 h, which was composed of 88 g of aqueous lower phase and 7 g of organic upper phase. The amount of condensate cannot be viewed as quantitative, as quantitative condensation cannot be assumed by dint of the nitrogen stream and the condensation temperature.

After 7 hours, the heating, the cooling, the magnetic stirrer and the nitrogen supply were deactivated. The reaction mixture thus cooled and remained in the reaction flask overnight.

The next morning, the reaction mixture was reheated to 40° C., since the melting point of the (possibly unreacted) dodecanol is 24° C.

The equimolar (with respect to the methanesulfonic acid) amount of sodium hydroxide (18.0 g) in the form of a 25% solution (72.0 g) was subsequently slowly added dropwise to the liquefied reaction mixture under vigorous stirring.

The reaction mixture largely neutralized in this way was transferred into a separatory funnel for phase separation and left in a drying cabinet at 40° C. until complete separation (2 to 4 h). After separation had taken place, 286 g of the product-bearing upper phase and 150 g of the aqueous/glycolic lower phase were obtained.

The chromatographic analysis of the product-bearing upper phase is shown in Table 2.

The identification of the detected substances was virtually quantitative (98.5%). Back-calculation of the recovery of the fatty alcohol-based reaction products quantified in the product-bearing upper phase, in relation to the amount of fatty alcohol used, yielded 100%. The calculated value of 102.4% is caused by the deviation of the semi-quantitative analysis in area %.

TABLE 2
Analysis of the worked-up upper phase

| | | | Conc. | Recovery |
|---|---|---|---|---|
| | | | [area | of FA |
| Component | | Category | %] | [mol %] |
| Ethylene glycol (MEG) | Polyol | Reactant | 0.30 | |
| 1-Dodecanol | Fatty | Reactant | 16.70 | 17.4 |
| | alcohol (FA) | | | |
| Diethylene glycol | MEG cond. | By-product | 0.19 | |
| Triethylene glycol | MEG cond. | By-product | 0.08 | |
| Tetraethylene glycol | MEG cond. | By-product | 0.11 | |
| Dodecyl monoethoxylate | FA | Target | 15.10 | 2.7 |
| | ethoxylate | product | | |
| Dodecyl diethoxylate | FA | Target | 3.10 | 4.4 |
| | ethoxylate | product | | |
| Dodecyl triethoxylate | FA | Target | 1.50 | 12.7 |
| | ethoxylate | product | | |
| Dodecyl tetraethoxylate | FA | Target | 0.65 | 1.4 |
| | ethoxylate | product | | |
| Dodecyl pentaethoxylate | FA | Target | 0.30 | 0.7 |
| | ethoxylate | product | | |
| Dodecyl hexaethoxylate | FA | Target | 0.14 | 0.4 |
| | ethoxylate | product | | |
| Dodecyl heptaethoxylate | FA | Target | 0.06 | 0.2 |
| | ethoxylate | product | | |
| Bisdodecyl ether | Bisalkyl | By-product | 52.5 | 57.6 |
| | ether | | | |
| Bisdodecyl ethylene | Bisalkyl | By-product | 5.90 | 2.9 |
| glycol | ether | | | |
| Bisdodecyl diethylene | Bisalkyl | By-product | 0.72 | 0.6 |
| glycol | ether | | | |
| Bisdodecyl triethylene | Bisalkyl | By-product | 0.19 | 0.2 |
| glycol | ether | | | |
| Bisdodecyl tetraethylene | Bisalkyl | By-product | 0.07 | 0.1 |
| glycol | ether | | | |
| Dodecene | Olefins | By-product | 0.97 | 1.1 |
| | | Reactants | 17.0 | 17.4 |
| | | Target | 20.8 | 22.5 |
| | | products | | |
| | | By-products | 60.7 | 62.5 |
| | | Total: | 98.5 | 102.4 |

### Working Example 3

Molar ratio of dodecanol, ethylene glycol, methanesulfonic acid=1+8+0.9; total charge 500 g.

120.8 g (0.65 mol) of dodecanol were weighed together with 57.2 g of methanesulfonic acid (0.58 mol) into a round-bottom flask and this was placed in a heating sleeve in a fume cupboard. Thereafter, this flask was equipped with a Liebig condenser (water temperature 20° C.), temperature sensor and a gas introduction pipe.

After activating a nitrogen stream of 0.5 l/min and heating (target temperature 150° C.), the reaction mixture was heated under vigorous stirring using a magnetic stirrer. The start of the reaction was defined upon reaching 100° C. From this point in time, a total of 322 g of ethylene glycol (5.19 mol) were added continuously (53.7 g/h) within the following 6 hours. The target temperature of 150° C. was reached after 15 min. After complete addition of the ethylene glycol (6 h), the reaction was continued further for an additional hour.

An amount of condensate of 259 g was detected gravimetrically over the entire reaction period of 7 h, which was composed of 221 g of aqueous lower phase and 38 g of organic upper phase. The amount of condensate cannot be viewed as quantitative, as quantitative condensation cannot be assumed by dint of the nitrogen stream and the condensation temperature.

After 7 hours, the heating, the cooling, the magnetic stirrer and the nitrogen supply were deactivated. The reaction mixture thus cooled and remained in the reaction flask overnight.

The next morning, the reaction mixture was reheated to 40° C., since the melting point of the (possibly unreacted) dodecanol is 24° C.

The equimolar (with respect to the methanesulfonic acid) amount of sodium hydroxide (23.8 g) in the form of a 25% solution (95.3 g) was subsequently slowly added dropwise to the liquefied reaction mixture under vigorous stirring.

The reaction mixture largely neutralized in this way was transferred into a separatory funnel for phase separation and left in a drying cabinet at 40° C. until complete separation (2 to 4 h). After separation had taken place, 110 g of the product-bearing upper phase and 167 g of the aqueous/glycolic lower phase were obtained.

The chromatographic analysis of the product-bearing upper phase is shown in Table 3.

The identification of the detected substances was virtually quantitative (96.5%). Back-calculation of the recovery of the fatty alcohol-based reaction products quantified in the product-bearing upper phase, in relation to the amount of fatty alcohol used, yielded approximately 88%. This reduced value compared to Working Example 2 shows that, under the conditions selected here, more fatty alcohol-based reaction products have remained in the aqueous/glycolic lower phase.

TABLE 3
Analysis of the worked-up upper phase

| | | | Conc. | Recovery |
|---|---|---|---|---|
| | | | [area | of FA |
| Component | | Category | %] | [mol %] |
| Ethylene glycol (MEG) | Polyol | Reactant | 0.06 | |
| 1-Dodecanol | Fatty | Reactant | 2.70 | 2.5 |
| | alcohol (FA) | | | |
| Diethylene glycol | MEG cond. | By-product | 0.03 | |
| Triethylene glycol | MEG cond. | By-product | 0.02 | |
| Tetraethylene glycol | MEG cond. | By-product | 0.12 | |
| Dodecyl monoethoxylate | FA | Target | 9.30 | 6.8 |
| | ethoxylate | product | | |
| Dodecyl diethoxylate | FA | Target | 3.10 | 3.8 |
| | ethoxylate | product | | |
| Dodecyl triethoxylate | FA | Target | 1.80 | 2.9 |
| | ethoxylate | product | | |
| Dodecyl tetraethoxylate | FA | Target | 0.62 | 1.2 |
| | ethoxylate | product | | |
| Dodecyl pentaethoxylate | FA | Target | 0.21 | 0.4 |
| | ethoxylate | product | | |
| Dodecyl hexaethoxylate | FA | Target | 0.18 | 0.4 |
| | ethoxylate | product | | |
| Dodecyl heptaethoxylate | FA | Target | 0.22 | 0.5 |
| | ethoxylate | product | | |
| Dodecyl octaethoxylate | FA | Target | 0.16 | 0.4 |
| | ethoxylate | product | | |
| Dodecyl nonaethoxylate | FA | Target | 0.09 | 0.2 |
| | ethoxylate | product | | |
| Dodecyl decaethoxylate | FA | Target | 0.05 | 0.1 |
| | ethoxylate | product | | |
| Bisdodecyl ether | Bisalkyl | By-product | 63.10 | 60.3 |
| | ether | | | |
| Bisdodecyl ethylene | Bisalkyl | By-product | 10.60 | 4.5 |
| glycol | ether | | | |
| Bisdodecyl diethylene | Bisalkyl | By-product | 1.60 | 1.2 |
| glycol | ether | | | |
| Bisdodecyl triethylene | Bisalkyl | By-product | 0.45 | 0.5 |
| glycol | ether | | | |
| Bisdodecyl tetraethylene | Bisalkyl | By-product | 0.17 | 0.2 |
| glycol | ether | | | |
| Bisdodecyl pentaethylene | Bisalkyl | By-product | 0.06 | 0.1 |
| glycol | ether | | | |
| Dodecene | Olefins | By-product | 1.90 | 1.9 |
| | | Reactants | 2.8 | 2.5 |
| | | Target | 15.7 | 16.9 |
| | | products | | |
| | | By-products | 78.0 | 69.0 |
| | | Total: | 96.5 | 88.4 |

### Working Example 4

Molar ratio of octadecanol, ethylene glycol, methanesulfonic acid=1+2+0.3; total charge 500 g

318.9 g (1.18 mol) of octadecanol were weighed together with 34.7 g of methanesulfonic acid (0.35 mol) into around-bottom flask and this was placed in a heating sleeve in a fume cupboard. Thereafter, this flask was equipped with a Liebig condenser (water temperature 20° C.), temperature sensor and a gas introduction pipe.

After activating a nitrogen stream of 0.5 l/min and heating (target temperature 150° C.), the reaction mixture was heated under vigorous stirring using a magnetic stirrer. The start of the reaction was defined upon reaching 100° C. From this point in time, a total of 146.4 g of ethylene glycol (2.36 mol) were added continuously (24.4 g/h) within the following 6 hours. The target temperature of 150° C. was reached after 15 min. After complete addition of the ethylene glycol (6 h), the reaction was continued further for an additional hour.

An amount of condensate of 78.5 g was detected gravimetrically over the entire reaction period of 7 h, which was composed of 76.2 g of aqueous lower phase and 2 g of organic upper phase. The amount of condensate cannot be viewed as quantitative, as quantitative condensation cannot be assumed by dint of the nitrogen stream and the condensation temperature.

After 7 hours, the heating, the cooling, the magnetic stirrer and the nitrogen supply were deactivated. The reaction mixture thus cooled and remained in the reaction flask overnight.

The next morning, the reaction mixture was reheated to 70° C., since the melting point of the (possibly unreacted) octadecanol is 59° C.

The equimolar (with respect to the methanesulfonic acid) amount of sodium hydroxide (14.4 g) in the form of a 25% solution (57.7 g) was subsequently slowly added dropwise to the liquefied reaction mixture under vigorous stirring.

The chromatographic analysis of the product-bearing upper phase is shown in Table 4.

TABLE 4
Analysis of the worked-up upper phase

| | | | Conc. | Recovery |
|---|---|---|---|---|
| | | | [area | of FA |
| Component | | Category | %] | [mol %] |
| Ethylene glycol (MEG) | Polyol | Reactant | 0.18 | |
| 1-Octadecanol | Fatty | Reactant | 33.30 | 34.3 |
| | alcohol (FA) | | | |
| Diethylene glycol | MEG | By-product | 0.23 | |
| | condensate | | | |
| Triethylene alycol | MEG | By-product | 0.07 | |
| | condensate | | | |
| Tetraethylene glycol | MEG | By-product | 0.05 | |
| | condensate | | | |
| Octadecyl | FA | Target | 9.70 | 8.6 |
| monoethoxylate | ethoxylate | product | | |
| Octadecyl diethoxylate | FA | Target | 1.40 | 2.2 |
| | ethoxylate | product | | |
| Octadecyl triethoxylate | FA | Target | 0.44 | 0.9 |
| | ethoxylate | product | | |
| Octadecyl tetraethoxylate | FA | Target | 0.19 | 0.5 |
| | ethoxylate | product | | |
| Bisoctadecyl ether | Bisalkyl | By-product | 46.10 | 49.1 |
| | ether | | | |
| Bisoctadecyl ethylene | Bisalkyl | By-product | 3.50 | 1.7 |
| glycol | ether | | | |
| Bisoctadecyl | Bisalkyl | By-product | 0.35 | 0.3 |
| diethylene glycol | ether | | | |
| Octadecene | Olefins | By-product | 0.97 | 1.1 |
| | | Reactants | 33.5 | 34.3 |
| | | Target | 11.7 | 12.2 |
| | | products | | |
| | | By-products | 51.3 | 52.2 |
| | | Total: | 96.5 | 98.7 |

The reaction mixture largely neutralized in this way was transferred into a separatory funnel for phase separation and left in a drying cabinet at 70° C. until complete separation (2 to 4 h). After separation had taken place, 328.5 g of the product-bearing upper phase and 115.5 g of the aqueous/glycolic lower phase were obtained.

The identification of the detected substances was virtually quantitative (96.5%). Back-calculation of the recovery of the fatty alcohol-based reaction products quantified in the product-bearing upper phase, in relation to the amount of fatty alcohol used, yielded approximately 99%.

### INDUSTRIAL APPLICABILITY

The invention proposes a process for preparing fatty alcohol ethoxylates which features, compared to the ethoxylation with ethylene oxide known from the prior art, the use of reactants with low hazardous material potential. According to the invention, the fatty alcohol ethoxylates are obtained here by etherification with ethylene glycol, an oligo ethylene glycol or a polyethylene glycol in the presence of an acidic catalyst.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1.-11. (canceled)

12. A process for preparing fatty alcohol ethoxylates, comprising reacting the fatty alcohol with ethylene glycol, an oligo ethylene glycol or a polyethylene glycol in the presence of an acidic catalyst.

13. The process according to claim 12, wherein a homogeneous acidic catalyst is used.

14. The process according to claim 13, wherein methanesulfonic acid is used as homogeneous acidic catalyst.

15. The process according to claim 13, wherein the reaction temperature is between 100 and 160° C.

16. The process according to claim 12, wherein the molar ratio of ethylene glycol to the fatty alcohol is between 0.1 and 10 mol/mol.

17. The process according to claim 16, wherein the reaction mixture is cooled after conducting the reaction and is neutralized by adding a base, a light, organic phase separating from a heavy, aqueous phase at the same time.

18. The process according to claim 17, wherein the light, organic phase is separated off from the heavy, aqueous phase by means of a phase separation apparatus.

19. The process according to claim 18, wherein the phase separation apparatus operates by the principle of sedimentation, centrifugation or decantation.

20. The process according to claim 18, wherein the light phase obtained after conducting the phase separation is worked up by means of a thermal separation process to obtain fatty alcohol ethoxylates.

21. The process according to claim 20, wherein the workup is performed by means of distillation or rectification.

22. The process according to claim 12, wherein an oligo ethylene glycol having a number of from 2 to 15 is used.
